(19) United States
(12) Patent Application Publication
(10) Pub. No.: US 2021/0330728 A1
(43) Pub. Date: Oct. 28, 2021

(54) Methods of Clinical Treatment of a Mammal Experiencing a Viral Respiratory Infection and/or Inflammation Resulting from a Viral Respiratory Infection Using Botanical Infusion

(71) Applicant: Jasmine Gardner, Ithaca, NY (US)

(72) Inventor: Jasmine Gardner, Ithaca, NY (US)

(21) Appl. No.: 17/237,145

(22) Filed: Apr. 22, 2021

Related U.S. Application Data
(60) Provisional application No. 63/013,661, filed on Apr. 22, 2020.

(51) Int. Cl. A61K 36/61; A61K 36/481; A61K 36/534; A61K 9/00; A61K 35/644; A61K 36/484; A61K 45/06; A61P 11/00

(52) U.S. Cl. CPC A61K 36/61; A61K 36/481; A61K 36/534; A61K 9/0053; A61K 35/644; A61K 36/484; A61K 2236/37; A61P 11/00; A61K 2236/331; A61K 2236/333; A61K 2236/17; A61K 2236/15; A61K 45/06

(57) ABSTRACT

As described herein, the invention includes methods for the treatment of a respiratory infection of a coronavirus and/or respiratory inflammation resulting from a coronavirus infection in a mammalian individual. The method includes administering to the individual an effective amount of a water-based infusion prepared by steeping the leaves of eucalyptus globulus Labill, the root of astragalus membranaceus, and the leaves of mentha X piperita in a liquid for a time duration and administering to the patient an effective amount of the infusion. Such administration may be via an oral or an inhalable route. Also included are methods of making a composition for use in the clinical treatment of a COVID-19 (SARS-CoV-2) infection and/or to treat inflammation resulting from an is COVID-19 (SARS-CoV-2) infection comprising steeping in a liquid leaves of eucalyptus globulus Labill, roots of astragalus membranaceus, and the leaves of mentha X piperita, for a duration of time.

### CROSS REFERENCE TO RELATED APPLICATION

This application claims the benefit under 35 U.S.C. § 119(e) of U.S. Provisional Patent Application Ser. No. 62/750,628, filed Apr. 22, 2020, entitled “Methods of Clinical Treatment of a Mammal Experiencing a Viral Respiratory Infection and/or Inflammation Resulting from a Viral Respiratory Infection Using Botanical Infusion,” the entire disclosure of which is incorporated herein by reference.

### BACKGROUND OF THE INVENTION

In December of 2019, an outbreak of pneumonia of unknown origin was identified in Wuhan city, Hubei province, China. By January 2020, it was confirmed that a new type of coronavirus named SARS-CoV-2 was responsible for these pneumonias. Named COVID-19 by the WHO, infected patients showed typical respiratory symptoms such as cough, fever, and lung damage in addition to other symptoms such as fatigue, myalgia, and diarrhea. As of Apr. 20, 2020, a total of 2,474,317 cases of the SARS-CoV-2 infection have been identified worldwide in at least 210 countries. Of these patients, 169,932 have died. Because of the rapid spread of SARS-CoV-2 through human-to-human transmission, entire countries have instituted nation-wide “stay at home” orders to stem the rate of transmission, resulting in significant economic damage worldwide.

SARS-CoV-2 is understood to be an enveloped single stranded RNA-type beta-coronavirus. The genome sequences of SARS-CoV-2 shared 79.5% sequence identity to severe acute respiratory syndrome-related coronaviruses (SARS-CoV). Infection occurs when the spike (S) protein of SARS-CoV-2 and SARS-CoV enters human alveolar epithelial cells through binding angiotensin- converting enzyme 2 (ACE2) receptor.

Unfortunately, no specific antiviral drugs or vaccines currently exist for treatment of COVID 19 infections. Thus, the treatment strategy for COVID-19 is supportive care, which is supplemented by the combination of broad-spectrum antibiotics, antivirals, corticosteroids and convalescent plasma. In addition to the know generalized treatments devised by modern medicine, application of so-called “traditional” treatments derived from botanical sources may be used to boost or supplement the activity of the patients' immune system and/or modern treatments. A need exists in the art for such botanical remedies, especially those targeted specifically to coronaviruses and more specifically to COVID-19.

### BRIEF SUMMARY OF THE INVENTION

The invention as described herein falls in the field of traditional medicine and medical botany. The purpose of the invention is to lessen the impact of coronavirus by removing fluid from the chest, stopping the virus from replicating in the lungs, repairing damaged lung cells, reducing inflammation and bolstering the immune system response. Each herb used within the botanical infusion of the invention has antiviral effects and at least one of these efficacies.

As described herein, the invention includes methods for the treatment of a respiratory infection of a coronavirus and/or respiratory inflammation resulting from a coronavirus infection in a mammalian individual. The method includes administering to the individual an effective amount of a water-based infusion prepared by steeping the leaves of eucalyptus globulus Labill, the root of astragalus membranaceus, and the leaves of mentha X piperita in a liquid for a time duration and administering to the patient an effective amount of the infusion. Such administration may be via an oral or an inhalable route. Also included are methods of making a composition for use in the clinical treatment of a COVID-19 (SARS-CoV-2) infection and/or to treat inflammation resulting from an is COVID-19 (SARS-CoV-2) infection comprising steeping in a liquid leaves of eucalyptus globulus Labill, roots of astragalus membranaceus, and the leaves of mentha X piperita, for a duration of time.

### DETAILED DESCRIPTION OF THE INVENTION

The invention described herein relates to a clinical treatment for a mammalian patient, preferable a human patient that can be used in the treatment of infections of viruses of the family Coronaviradae, especially the specific virus called COVID-19 which apparently emerged in late 2019 or early 2020. “Treatment” as used herein includes the actual inactivation or elimination of the virus (by for example, inhibition or interference of the virus at the attachment and penetration stages, inhibition or interference of necessary viral processes, such as viral replication, protein synthesis, etc.) and/or of the respiratory infection as well as elimination or amelioration of any direct or indirect symptoms caused by infection, such as inflammation, cough, sore throat, respiratory inflammation, etc.

As used herein, the measurement “tablespoon”, “teaspoon”, and “cup” refer to the Queen Anne (US) measurement system.

The method includes administering to the patient an infusion that is prepared from the leaves of eucalyptus globulus Labill, the root of astragalus membranaceus, and the leaves of mentha X piperita (collectively, “the botanicals”). The infusion may be prepared by steeping the botanicals in a liquid, preferably a water-based liquid (e.g., more than 50% water by total weight), but other solvents or extractants may be included, such as for example, ingestible alcohols or glycerin.

The botanicals may be steeped at any temperature. Room temperature may be preferred. In an embodiment, one may apply heat to the botanical/liquid mixture for all or a part of the steeping step. As an example, one may steep the botanical/liquid mixture for a total of 4 hours but may apply heat for 1 hour or for the entire four hours.

In an embodiment the duration of the steeping step is about 10 minutes to about 10 hours, about 1 hour to about 7 hours, or about 3 hours to about 5 hours. If heat is applied for any portion of this step, in an embodiment it is applied for about 30 minutes to about 60 minutes. Such heat may be applied at temperatures of about 90° C. to about 110° C. for at least a portion of the time duration. In an embodiment, the preferred temperature is about 100° C.

As noted above, the botanicals included in the infusion are leaves of eucalyptus globulus Labill (“eucalyptus”), the root of astragalus membranaceus (“astragalus”, and the leaves of mentha X piperita (“peppermint”). The roots/leaves may independently be in any form, e.g., fresh, dried, frozen, pickled, or otherwise preserved. They may be in any format, for example, whole, chopped, cut, crumbled, comminuted, shredded, powdered, pulverized, etc.

In an embodiment, the botanicals are dried and in powdered or crumbled form.

The botanicals may be sourced from any region or area from which they are or will be available. Organically grown botanicals may be preferred. In an embodiment, the eucalyptus is sourced from Portugal, the astragalus is sourced from China, and the peppermint was grown in the United States.

In an embodiment, the relative amount of the botanicals may be as follows (per about 7 to about 10 quarts of liquid):

| Botanical | Amount |
|---|---|
| Eucalyptus | about 1 to about 5 tablespoons |
| | about 2 to about 3.5 tablespoons |
| | about 1.5 tablespoons |
| Astragalus | about 2 to 7 tablespoons |
| | about 3.5 to about 4 tablespoons |
| | about 2.5 tablespoons |
| Peppermint | about 4 to about 9 tablespoons |
| | about 6 to about 9 tablespoons |
| | 5.3 tablespoons (1/3 cup) |

Other ingredients may be included in the infusion for clinical, palliative or cosmetic reasons. Examples might include a sweetener, honey, raw honey, a vitamin, an antioxidant, an expectorant, a topical anesthetic, a cough suppressant, rosemary, curcumin, marshmallow root, comfrey, chamomile, garlic, a licorice, an anti-inflammatory compound, an antihistamine, a preservative, an amino acid, a colorant, an opacifier and a flavorant.

### EXAMPLE 1

A male in his mid-twenties engaged in extensive travel with a high level of social interaction in the last few week of February and beginning of March 2020. He returned to his home in Ithaca, NY, United States, on Mar. 8, 2020 suffering from chills and a sore throat. Within 24 hours he developed a dry cough with fever and severe fatigue. As the day progressed, he complained of chest tightness and difficulty breathing. An infusion was prepared as follows: 1.5 tablespoons of dried and crumbled eucalyptus leaves, 2.5 tablespoons of dried and powdered astragalus and ⅓ cup of dried and crumbled peppermint leaves were placed in 7 quarts of water to steep. The water/botanicals mixture was heated to an observable boil, then boiled for about 50 minutes with occasional stirring.

The heat was them removed and the mixture was permitted to continue steeping for about another 3 hours. The remains of the botanicals were removed from the infusion by mechanical straining.

The male patient was orally administered a 4-ounce dosage of the infusion every about three hours over a 24 hour period after which he reported that his ability to breathe had improved by about 90 to 95%.

### EXAMPLE 2

A female in her mid-twenties lives in the same household as the male of Example 1 (her husband). Within a week of the male's return to the household, the female developed a fever, chills and a cough. An infusion was prepared as follows: 1.5 tablespoons of dried and crumbled eucalyptus leaves, 2.5 tablespoons of dried and powdered astragalus and ⅓ cup of dried and crumbled peppermint leaves were placed in 7 quarts of water to steep. The water/botanicals mixture was heated to an observable boil, then boiled for about 50 minutes with occasional stirring.

The heat was them removed and the mixture was permitted to continue steeping for about another 3 hours. The remains of the botanicals were removed from the infusion by mechanical straining.

The infusion is heated again to boiling in a tented heat proof container so that steam is generated from the infusion and remains in the “tent”. The female patient's head is placed in the tent and allowed to breathe normally the steam infused air. This treatment may be repeated every 2 to 3 hours until relief is experienced.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A method for the treatment of a respiratory infection of a coronavirus and/or respiratory inflammation resulting from a coronavirus infection in a mammalian individual comprising administering to the individual an effective amount of a water-based infusion prepared by steeping the leaves of eucalyptus globulus Labill, the root of astragalus membranaceus, and the leaves of mentha X piperita in a liquid for a time duration and orally administering to the patient an effective amount of the infusion.

2. The method of claim 1 wherein the liquid is 100% water.

3. The method of claim 1 wherein the liquid is a mixture of water and an alcohol.

4. The method of claim 1 wherein the leaves or root of each of the eucalyptus globulus Labill, astragalus membranaceus, and mentha X piperita are independently in a dried form.

5. The method of claim 1 wherein the leaves or root of each of the eucalyptus globulus Labill, astragalus membranaceus, and mentha X piperita are independently in a form selected from powdered, pulverized, crumbled, and comminuted.

6. The method of claim 1 wherein
the eucalyptus globulus Labill is present in amount of about 1 to about 2 tablespoons,
the astragalus membranaceus is present in amount of about 2 to 3.5 tablespoons, and
the mentha X piperita is present in an amount of about 4 to about 6 tablespoons, per about 7 quarts of liquid.

7. The method of claim 1 wherein
the eucalyptus globulus Labill is present in amount of about 1.5 tablespoons,
the astragalus membranaceus is present in amount of about 2.5 tablespoons, and
the mentha X piperita is present in an amount of about 5.3 tablespoons (⅓ cup), per about 7 quarts of liquid.

8. The method of claim 1 wherein the time duration of steeping is about 10 minutes to about 10 hours.

9. The method of claim 1 wherein steeping further comprises application of heat of a temperature of about 90° C. to about 110° C. for at least a portion of the time duration.

10. The method of claim 9 wherein the heat is applied for about 30 minutes to about 60 minutes.

11. The method of claim 1 wherein the individual is orally administered the infusion in an amount of about 3 ounces to about 40 ounces.

12. The method of claim 1 wherein the individual is a human.

13. The method of claim 1 wherein the infusion further comprises at least one additional ingredient selected from a sweetener, honey, a vitamin, an antioxidant, an expectorant, a topical anesthetic, a cough suppressant, a licorice, an anti-inflammatory compound, an antihistamine, a colorant, an opacifier and a flavorant.

14. The method according to claim 1, wherein the corona virus is COVID-19 (SARS-CoV-2).

15. The method of claim 1 wherein the leaves or roots are removed from the infusion prior to the administration of the infusion.

16. A method of making a composition for use in the clinical treatment of a COVID-19 (SARS-CoV-2) infection and/or to treat inflammation resulting from an is COVID-19 (SARS-CoV-2) infection comprising steeping in a liquid
leaves of eucalyptus globulus Labill,
roots of astragalus membranaceus,
leaves of mentha X piperita,
for a duration of time.

17. The method of claim 16 wherein the leaves or root of each of the eucalyptus globulus Labill, astragalus membranaceus, and mentha X piperita are independently in a dried form.

18. The method of claim 16 wherein the leaves or root of each of the eucalyptus globulus Labill, astragalus membranaceus, and mentha X piperita are independently in a form selected from powdered, pulverized, crumbled, and comminuted.

19. The method of claim 16 wherein
the eucalyptus globulus Labill is present in amount of about 1 to about 2 tablespoons,
the astragalus membranaceus is present in amount of about 2 to 3.5 tablespoons, and
the mentha X piperita is present in an amount of about 4 to about 6 tablespoons, per about 7 quarts of liquid.

20. The method of claims 16 wherein
the eucalyptus globulus Labill is present in amount of about 1.5 tablespoons,
the astragalus membranaceus is present in amount of about 2.5 tablespoons, and
the mentha X piperita is present in an amount of about 5.3 tablespoons (⅓ cup), per about 7 quarts of liquid.

21. The method of claim 16 wherein the time duration of steeping is about 10 minutes to about 10 hours.

22. The method of claim 16 wherein steeping further comprises application of heat of a temperature of about 90° C. to about 110° C. for at least a portion of the time duration.

23. The method of claim 22 wherein the heat is applied for about 30 minutes to about 60 minutes.

24. The method of claim 16 further comprising incorporating into the infusion at least one of a sweetener, honey, a vitamin, an antioxidant, an expectorant, a topical anesthetic, a cough suppressant, a licorice, an anti-inflammatory compound, an antihistamine, a colorant, an opacifier and a flavorant.
